(19) United States
(12) Patent Application Publication
(10) Pub. No.: US 2009/0297591 A1
(43) Pub. Date: Dec. 3, 2009

(54) Compositions And Methods For The Transdermal Delivery Of Pharmaceutical Compounds

(75) Inventors: Chin-Chih Chiang, West Covina, CA (US); Tse-Ching Lin, Taipei County (TW); Remy Chen, Taichung City (TW)

Correspondence Address: MORRIS MANNING MARTIN LLP, 3343 PEACHTREE ROAD, NE, 1600 ATLANTA FINANCIAL CENTER, ATLANTA, GA, 30326, US

(73) Assignee: Orient Pharma Co., Ltd., Taipei (TW)

(21) Appl. No.: 12/465,923

(22) Filed: May 14, 2009

Related U.S. Application Data
(60) Provisional application No. 61/129,015, filed on May 30, 2008.

(51) Int. Cl. A61K 9/70; A61K 31/167; A61K 31/24; A61K 31/27; A61K 31/137; A61P 43/00

(52) U.S. Cl. 424/449; 514/626; 514/534; 514/490; 514/654

(57) ABSTRACT

The present invention is directed to compositions and methods for the transdermal delivery of a pharmaceutically active compound. In some embodiments, the addition of inert pharmaceutical ingredients in place of a portion of adhesive in a transdermal patch formulation increases the rate of skin permeation of a pharmaceutical compound.

This application claims the benefit of U.S. Provisional Application No. 61/129,015 filed May 30, 2008, which is incorporated herein by reference.

### BACKGROUND

Transdermal delivery of pharmaceutically active ingredients can be done using transdermal patches with adhesive matrices. Transdermal patches can be used to treat a variety of diseases after having been applied onto the skin. Transdermal patches can be composed of three layers: a) a backing layer, b) a drug/adhesive layer, and c) a release liner.

Before applying a transdermal patch to a recipient's skin, the release liner has to be peeled off to expose the drug/adhesive layer. The drug/adhesive layer is firmly adhered on the backing layer. The drug/adhesive layer will then be put on the skin with a little pressure on the backing layer to make sure the adhesive is firmly adhered onto the skin.

The usefulness of transdermal patch formulations is often reduced by poor permeation of the active ingredient across the skin. Skin permeation of pharmaceutically active ingredients is primarily controlled by the stratum corneum, the outer most skin layer. Skin permeation at steady state is best described by Fick's first law,

J=P×C/h, Equation 1

where J is the steady state permeation flux, P is the permeability of the permeant, C is the initial concentration and h is the thickness of skin.

The poor permeability of skin often precludes the pharmaceuticals from penetrating through a patient's skin to provide a sufficient in vivo concentration to achieve the desired pharmacological response. Skin penetration can be enhanced by, for example: a. increasing skin permeability for drug; b. increasing drug diffusivity in the skin; c. increasing drug solubility in the skin. Such techniques can allow a drug candidate to permeate through a patient's skin and enter into blood stream. This improves the drug candidate's efficacy and making it a potential candidate to be delivered transdermally.

Various patents and literature have discussed the incorporation of skin permeation enhancers to increase permeability of drugs in the skin. These enhancers penetrate into skin to reversibly decrease the barrier resistance. Numerous compounds have been evaluated for penetration enhancing activity, including dimethylsulphoxide (DMSO), Azones (n-dodecyl-cyclazacycloheptan-2-one), 2-pyrrolidone, ethanol, decanol, propylene glycol, surfactants and terpenes. Many potential sites and modes of action have been identified for skin penetration enhancers; such as the intercellular lipid matrix in which the acceletants may disrupt the packing motif, the intracellular keratin domains or through increasing drug partitioning into the tissue by acting as a solvent for the permeant within the membrane.

Patents and literature have also discussed the incorporation of chemicals to increase solubility of drugs in patch and skin. For example, compounds such as propylene glycol, ethanol, and combination of lauryl lactate and lauryl glycol can be used as solubilizers.

However, since skin permeation enhancers may cause skin irritation, there is a need for transdermal patches with high rates of skin permeation that use alternative methods of increasing skin permeation.

### SUMMARY

The present invention is directed to compositions and methods for the transdermal delivery of a pharmaceutically active compound. In some embodiments, the addition of inert pharmaceutical ingredients in place of a portion of adhesive in a transdermal patch formulation increases the rate of skin permeation of a pharmaceutical compound.

Particularly, a transdermal patch includes a backing layer; an adhesive drug matrix having a pharmaceutically active ingredient and at least one pharmaceutically inactive ingredient; and a release liner. A method for preparation of patch includes mixing a predetermined amount of least one pharmaceutically active ingredient with predetermined amount at least one pharmaceutically inactive ingredient to form a mixture; adding the mixture into a solution with an adhesive; mixing the solution until it becomes homogenous; film coating a release liner with the homogeneous solution; and laminating the film coated release liner to produce a transdermal patch.

The foregoing, as well as additional objects, features and advantages of the invention will be more readily apparent from the following detailed description, which proceeds with reference to the accompanying drawings.

### BRIEF DESCRIPTION OF THE DRAWINGS

The present invention will be discussed in more detail below, using a number of exemplary embodiments, with reference to the attached drawings, in which:

FIG. 1 illustrates the skin permeation profiles of lidocaine from patches of an example embodiment of the present invention and a commercially available product, Lidoderm. () Lidoderm; (∘) Formulation of Example 4;

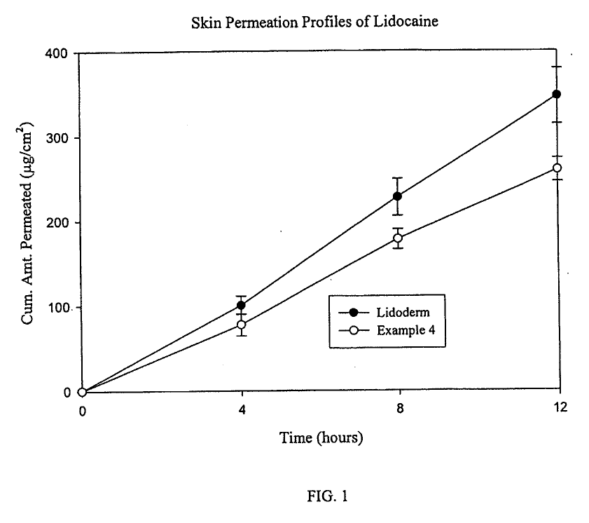

FIG. 2 illustrates the skin permeation profiles of lidocaine from patches of an example embodiment of the present invention and a commercially available product, Lidoderm. () Lidoderm; (∘) Formulation of Example 10;

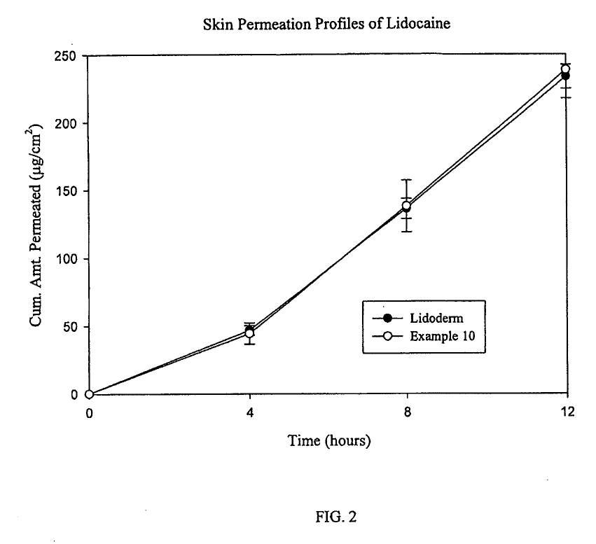

FIG. 3 illustrates the skin permeation profiles of lidocaine from patches of an example embodiment of the present invention and a commercially available product, Lidoderm. () Lidoderm; (∘) Formulation of Example 15; and

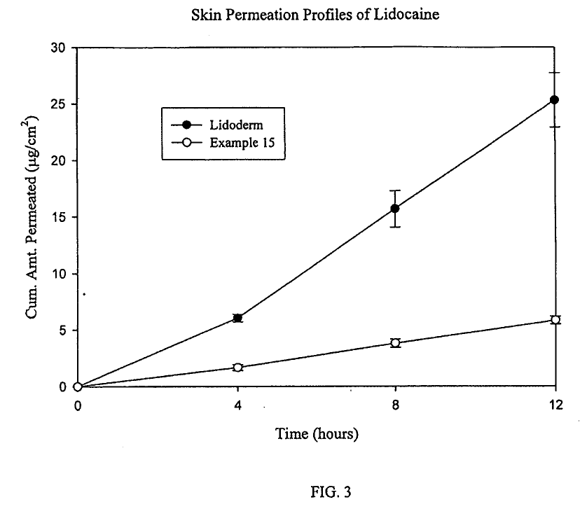

FIG. 4 illustrates the skin permeation profiles of lidocaine from patches of an example embodiment of the present invention and a commercially available product, Lidoderm. ()Lidoderm; (∘) Formulation of Example 18.

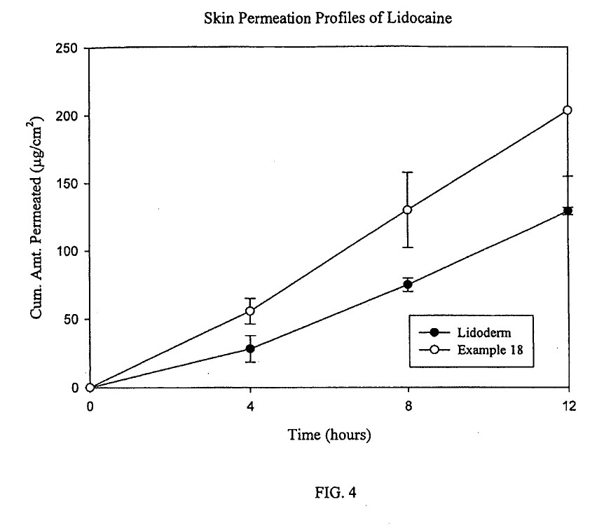

### DETAILED DESCRIPTION OF EXEMPLARY EMBODIMENTS

Embodiments of the invention are directed to compositions and methods for the transdermal delivery of pharmaceutically active compounds. It has surprisingly been found that the addition of inert pharmaceutical ingredients in place of a portion of the adhesive in a transdermal patch formulation increases the rate of skin permeation of these pharmaceutical compounds.

As used herein, the term “transdermal patch” refers to a medicated adhesive composition that can be applied to the skin to deliver a dose of the medication locally and/or into the bloodstream. In some embodiments, a transdermal patch has three layers: a backing layer, a drug/adhesive matrix, and a release layer.

The backing layer can be made of any suitable material that is impermeable to the components used in the drug/adhesive matrix and is capable of protecting the patch from the environment when applied to the skin. Suitable materials for the backing layer include, but are not limited to, commercially available films of polyester film laminate such as Scotchpak 9733 backing film sold by 3M.

In some embodiments, the release liner can be a polyester film coated on one side with Teflon™ or silicon for ease of separation from adhesive. The release liner can be peeled off from the drug/adhesive layer to expose the drug/adhesive layer prior of the transdermal patch before affixing the patch onto skin. In some embodiments, the materials for making the release liner are commercially available films of fluoropolymer coated polyester film such as Scotchpak 1022 sold by 3M.

The drug/adhesive matrix can contain the pharmaceutically active compound incorporated into at least one adhesive. The pharmaceutically active ingredient can be dissolved, dispersed, suspended or otherwise distributed within the adhesive. In some embodiments, the pharmaceutically active ingredient is homogeneously dispersed within the adhesive. The drug/adhesive matrix can be sandwiched between the release liner and backing layer to construct a transdermal patch.

Suitable adhesives include, but are not limited to, polyisobutylene, polyacrylate, silicone elastomers, and combinations thereof. In some embodiments, the adhesive is polyisobutylene, a polyacrylate or a silicone elastomer. Polyacrylate is commercially available as Gelva 737, Gelva 788 from Cytec or Duro-Tak solutions such as Duro-Tak 87-2852 or Duro-Tak 87-2287 from National Starch and Chemicals. These adhesives are available as solutions. The organic solvents, which are used to dissolve the adhesives, have to be evaporated during the manufacturing procedures.

In some embodiments, the adhesive is a pressure sensitive adhesive. For example, a suitable pressure sensitive adhesive for the patch is polyacrylate polymer. Polyacrylate adhesive is used to help the patch stay on the skin for a desired period of time. As one of skill in the art will appreciate, other suitable pressure sensitive adhesives can be used in the present invention as well.

The adhesive of the drug/adhesive layer can also contain inactive ingredients. The addition of inactive ingredients, in place of a portion of the adhesive has been surprisingly found to increase the ability of the active ingredient to penetrate the skin. In embodiments of the present invention, chemically and pharmaceutically inert materials can be incorporated into transdermal patches to replace a portion of adhesive, thereby increasing the concentration of a pharmaceutically active ingredient in the adhesive. This has been found to result in higher skin permeation rate of pharmaceutically active ingredients.

According to the Fick's first law (Equation 1 above), the permeation flux is proportional to the initial concentration of permeant. In the development process of patch formulations, intentions are made to reach the maximum concentrations of pharmaceutically active ingredients in the carrier, e.g., the adhesive, as high as possible. If the solubility of pharmaceutically active ingredient in the adhesive is too high, a high quantity of the active ingredient has to be added to reach the highest degree of saturation. In this case, not only a high quantity of drug has to be added, a big portion of the active ingredient will remain in the adhesive which can not be delivered during a certain period of time and becomes a waste.

In embodiments of the current invention, pharmaceutically inactive ingredients are added to replace a portion of adhesive. The pharmaceutically inactive ingredients can be added and dispersed into the adhesive solution, often homogeneously. The mixture can be coated on a polyester film and dried to evaporate the organic solvents which are used to dissolve the adhesive.

The adhesive is a continuous phase and the pharmaceutically inactive ingredients are a discontinuous phase. The pharmaceutically inactive ingredients have no substantial interaction with the pharmaceutically active ingredients. The concentration of pharmaceutically active ingredients in the continuous phase, adhesive, is increased. Since C (concentration in Equation 1) of pharmaceutically active ingredient in the adhesive is increased with the same loading, the skin permeation of the pharmaceutically active ingredient is increased.

In some embodiments, the pharmaceutical inactive ingredient can be selected from talc, magnesium stearate, titanium dioxide, starch, silicon dioxide or sorbitol. The amount of silicon dioxide can be about 0.2% to about 5.0% by weight. As used herein, “about” refers to plus or minus 10% of the indicated number.

Preferably, the amount of the silicon dioxide is in the range of 1.0 to 3.0% by weight. The amount of other pharmaceutically inactive ingredients is in the range of 20-75% by weight. Preferably, the amount of other pharmaceutically inactive ingredients is in the range of 40-65% by weight. In some embodiments, the percentages used herein are by weight of the amount of the adhesive/drug matrix.

The “pharmaceutically active ingredient” can be a drug, vitamin, or other pharmaceutically acceptable composition that exerts at least one desired therapeutic effect when administered to a mammal in need thereof, e.g., a human. The pharmaceutically active ingredient, in some embodiments, has a high solubility in the adhesive used in the transdermal patch.

In some embodiments, the pharmaceutically active ingredient is a chemical compound in its base form. Chemicals in their base forms can be oils or exist in crystal forms and may show high solubilities in the adhesive (e.g., a polyacrylate). For example, lidocaine and oxybutynin in their base forms show low melting points of 68 and 57° C., respectively. Rivastigmine base is an oil at room temperature. Tolterodine in its base form is a viscous material at room temperature. Their solubilities in polyacrylate adhesives are greater than 10%. Accordingly, in some embodiments, these chemicals can be delivered transdermally using the compositions and methods disclosed herein. In some embodiments, the drug used in the drug adhesive matrix has a low melting point.

Accordingly, in some embodiments, the pharmaceutically active ingredient is lidocaine, tolterodine, oxybutynin, or rivastigmine, all of which are soluble in the adhesive. The amount of the pharmaceutically active ingredient in the adhesive can be in the range of about 1% to about 10% by weight. More preferably, the amount of the pharmaceutically active ingredient in the adhesive is in the range of about 3% to about 6% by weight.

The pharmaceutically active ingredient can also be, but is not limited to, analgesics, anti-inflammatories, antipsychotics, antipyretics, antibiotics, antimicrobials, anorexics, antihistamines, antiasthmatics, antidiuretics, antimigraine agents, antispasmodics, sedatives, antihyperactives, antihypertensives, tranquilizers, decongestants, beta blockers and combinations thereof. Additional representative examples include anti-inflammatory agents such as aceclofenac, diclofenac, flubiprofen, sulindac and celecoxib; analgesics such as acetaminophen and aspirin; agents for erectile dysfunction therapy such as sildenafil and apomorphine; anti-migraine agents such as, sumatriptan and ergotamin; anti-cholinergic agents, scopolamine hydrobromide; the antihistaminic agents, loratadine, fexofenadine and cetirizine; the cardiovascular agents, nitroglycerine and isosorbide dinitrate; the diuretics, furocemide and spironolactone; the anti-hypertensive agents, nimodipine, propranolol, amlodipine, felodipine, nifedipine, captoprile, ramiprile, atenolol and diltiazem; the anti-hypolipidemic agents, lovastatin, simvastatin, atrovastatin and pravastatin; the anti-ulcer agents, cimetidine, ranitidine, famotidine, omeprazole and lansoprazol; the anti-emetics, meclizine hydrochloride, ondansetron, granisetron, ramosetron and tropisetron; the anti-asthmatic agents, aminophylline, theophylline, terbutaline, fenoterol, formoterol and ketotifen; the anti-psychotics, clonazepam, olanzapine and risperidone; the anti-depressants, mirtazapine, fluoxetine and sertraline; the vitamins, B1, B2, B6, B12 and C; the anti-thrombotic agents, sulfinpyrazone, dipyridamole and ticlopidine; the chemotherapeutic agents, cefaclor, bacanpicillin, sulfamethoxazole and rifampicin; the hormones, dexamethasone and methyltestosterone; the anthelmintic agents, piperazine, ivermectine and mebendazole; and the anti-diabetic agents, acarbose, gliclazid and glipizid; drugs useful for Alzheimer's, memantin, donepezil, galantamine, galantamine hydrobromide, rivastigmine; drugs useful for Parkinson's disease, pramipexole; drugs useful for pain management, alprazolam, tamsolosin, alfuzosin, fentanyl; hormones, cyproterone acetate, oxadralone; antihypertensives, clonidine; psychostimulants, modafinil; drugs for relief of heartburn, lanzoprazole. As one of skill in the art will appreciate, any of these compounds may be used in the trandermal patches described herein, provided the compound can be successfully dispersed in the adhesive.

In some embodiments, the ratio of the adhesive in the drug/adhesive matrix to the amount of pharmaceutically active ingredient is from about 9:1 to about 9.9:0.1 (w/w). For example, the ratio can be about 9:1, about 9.5:0.5 or about 9.9:0.1 (w/w).

In one embodiment, the transermal patch comprises a backing layer of water insoluble material, an adhesive drug matrix, and a release liner. The adhesive drug matrix contains a pharmaceutical ingredient, such as lidocaine; a pharmaceutically inactive ingredient, and a pressure sensitive adhesive.

In another embodiment, the pharmaceutically inactive and inert ingredients are added to the pressure sensitive adhesives. The pharmaceutically active ingredients are highly soluble in the adhesives. In the present invention the pharmaceutically inactive or inert ingredients are added to replace a portion of the adhesive.

Lidocaine has been used as a model drug in tests demonstrating the benefits of some embodiments of the invention described herein. Lidoderm, which is commercially available product having 5% (w/w) lidocaine loading, is used as a reference drug.

Lidoderm is comprised of an adhesive material containing 5% lidocaine, which is applied to a non-woven polyester felt backing and covered with a polyethylene terephthalate film release liner. Each adhesive patch contains 700 mg of lidocaine and other pharmaceutically inactive ingredients in an aqueous base.

The current invention has been formulating lidocaine in polyacrylate adhesives. Lidocaine concentration in the adhesive is fixed at 5% (w/w). However, the solubility of lidocaine in polyacrylate is high, greater than 10% (w/w). Lidocaine tends to stay in the adhesive in stead of releasing to the skin, resulting in lower skin permeation. The incorporation of inactive ingredients such as talc (a mineral composed of hydrated magnesium silicate with the chemical formula H₂Mg₃(SiO₃)₄ or Mg₃Si₄O₁₀(OH)₂) or TiO₂ (titanium dioxide) to replace a portion of adhesive increases the lidocaine concentration in the adhesive or C value in the equation, thus resulting in a higher skin permeation rate of lidocaine.

As it is shown in the skin permeation studies in the Examples below, lidocaine from Lidoderm and from Example 4 shows skin permeation rates of 206.96±31.37 and 111.36±1.79 μg/cm² in 12 hours, respectively. Skin permeation of lidocaine from Lidoderm is 1.86 fold as high as that from Example 4. However, for Examples 18, 19 and 20 in which TiO2 was added, skin permeation rates of lidocaine from those examples are 203.45±48.37, 215.59±20.31 and 250.22±88.12 μg/cm² in 12 hours, respectively. In the same study, the skin permeation of lidocaine from Lidoderm is 129.36±2.78 μg/cm² in 12 hours. The skin permeation of lidocaine in those patches where in TiO2 was added shows 1.57, 1.67 and 1.94 fold as high as compared to Lidocaine.

Embodiments of the present invention are also directed to methods of preparing the transdernal patches disclosed herein. For example, appropriate amounts of pharmaceutically active and inactive ingredients, are accurately weighed and added to at least one vessel. These ingredients are then dissolved or suspended in a solution containing the adhesive. The mixture can be stirred until the solution is a homogeneous mixture.

A sheet of release liner is placed onto a patch coater (e.g., Warner Mathis coater). After becoming homogeneous, the solution is poured onto the release liner to form a thin film coat on the release liner. The coated release liner is dried in an oven at a temperature of about 40-80° C., for example 60° C., for about 5-30 minutes, for example 10 minutes to evaporate the solvent.

After drying, the dried film coated release liner is laminated with a sheet of backing layer. This laminate is cut using, for example, a die cutter into desired sizes to produce transdermal patches.

The present invention is also directed to methods of treating a disease or disorder using the transdermal patches described herein. For example, the patches may be applied to a mammal in need of treatment to deliver a predetermined dose of the pharmaceutically active ingredient over a predetermined time period. In some embodiments, the patches can be administered to a human to treat Alzheimer's disease, Parkinson's disease, to provide pain relief (e.g., from either chronic or breakthrough pain), and/or to treat hypertension, depending on the active ingredient in the patch.

All of the various embodiments or options described herein can be combined in any and all variations. The following Examples serve only to illustrate the invention and are not to be construed in anyway to limit the invention.

### EXAMPLE 1

Preparation of the Transdermal Patches:
1. Weigh appropriate amounts of pharmaceutically active and inactive ingredients and adhesive solutions, accurately weighed in a vessel.
2. Dissolve or suspend the ingredients in the adhesive solution and mix the solution until homogeneous.
3. Place a sheet of release liner onto a patch coater (e.g., Warner Mathis coater).
4. Pour the solution on the release liner and coat a thin film on the release liner.
5. Dry the solution in an oven which is preset at temperatures of 60° C. and drying time of 10 minutes to evaporate the solvent.
6. After drying, laminate the dried film with a sheet of backing layer.
7. Cut the laminate with die cutter into desired sizes.

### EXAMPLE 2

In Vitro Skin Permeation Studies

The lidocaine patch as described in Example 1 is evaluated to determine the skin permeation of lidocaine. Lidoderm is included in the study for comparison purposes. Lidoderm is a patch of 10×14 cm in size each having 5% lidocaine loading (or 700 mg). Lidoderm was cut into patches of 5 cm² each.

The in-vitro permeation of lidocaine through human cadaver skin was performed using VC skin diffusion cells. The active permeation area was 0.64 cm². Human cadaver skin was cut to a desired size and placed on a flat surface of one VC skin diffusion cell with the stratum corneum side facing outward. The release liner was separated from the polyacrylate drug matrix. The drug matrix was placed onto the stratum corneum. The above was repeated for another set of VC skin diffusion cell. The two sets were then clamped together. 20% Polyethylene glycol in distilled water solution of 3.5 mL was added to the receptor site of the diffusion cell to initiate the skin permeation study. Temperature of distilled water was maintained at 37° C. by circulating water from a water bath.

At predetermined time intervals, i.e., 4 hr, 8 hr and 12 hr, 0.5 mL each of receptor vehicle was withdrawn. After withdrawal fresh vehicle was added to maintain the same volume of 3.5 mL at each time. Lidocaine concentration in the samples was assayed by an HPLC. The cumulative amount of lidocaine in the receptor compartment as a result of skin permeation was calculated and reported. The following table shows the amount of lidocaine delivered as results of skin permeation study.

### EXAMPLE 3

HPLC Assay (Lidocaine):

Lidocaine concentrations in skin permeation samples are assayed by HPLC methods. The HPLC conditions are listed as follows.
Column: C18, 250× 4.6 mm, 5μ
Mobile Phase: 36% Methanol and 64% 0.1 M Phosphate Buffer (pH 3.0) with 0.1% Triethanolamine.
Wavelength: 210 nm
Volume of Injection: 10 μL

HPLC Assay (Tolterodine):

Tolterodine concentrations in skin permeation samples are assayed by HPLC methods. The HPLC conditions are listed as follows.
Column: C18, 250× 4.6 mm, 5μ
Mobile phase: 46% Methanol and 54% 0.1 M Phosphate Buffer (pH 3.0) with 0.1% Triethanolamine.
Wavelength: 230 nm
Volume of Injection: 10 μL

### EXAMPLE 4

An adhesive matrix composed of lidocaine 5.0% (w/w), propylene glycol 10.0% (w/w) and Gelva 737 adhesive solution (32.3% polyacrylate) was prepared according to the manufacturing procedures as described in Example 1. Cumulated amount of lidocaine from Example 4 in the in-vitro skin permeation study during 12 hours was 111.36±1.79 (1.6%) μg/cm².

### EXAMPLE 5

An adhesive matrix composed of lidocaine 5.0% (w/w), propylene glycol 8.0% (w/w) and Gelva 737 adhesive solution (32.3% polyacrylate) was prepared according to the manufacturing procedures as described in Example 1. Cumulated amount of lidocaine from Example 5 in the in-vitro skin permeation study during 12 hours was 124.24±1.79 (1.4%) μg/cm².

### EXAMPLE 6

An adhesive matrix composed of lidocaine 5.0% (w/w), Isopropyl myristate 10.0% (w/w) and Gelva 737 adhesive solution (32.3% polyacrylate) was prepared according to the manufacturing procedures as described in Example 1. Cumulated amount of lidocaine from Example 6 in the in-vitro skin permeation study during 12 hours was 377.47±36.78 (9.7%) μg/cm².

### EXAMPLE 7

An adhesive matrix composed of lidocaine 5.0% (w/w), Isopropyl Myristate 15.0% (w/w), and Gelva 737 adhesive solution (32.3% polyacrylate) was prepared according to the manufacturing procedures as described in Example 1. Cumulated amount of lidocaine from Example 7 in the in-vitro skin permeation study during 12 hours was 386.93±18.89 (4.9%) μg/cm².

### EXAMPLE 8

An adhesive matrix composed of lidocaine 5.0% (w/w), Isopropyl Myristate 5.0% (w/w), Propylene Glycol 5.0% and Gelva 737 adhesive solution (32.3% polyacrylate) was prepared according to the manufacturing procedures as described in Example 1. Cumulated amount of lidocaine from Example 8 in the in-vitro skin permeation study during 12 hours was 232.67±19.37 (8.3%) μg/cm².

### EXAMPLE 9

An adhesive matrix composed of lidocaine 5.0% (w/w), propylene glycol 5.0% (w/w), Tween-80 5.0% (w/w) and Gelva 737 adhesive solution (32.3% polyacrylate) was prepared according to the manufacturing procedures as described in Example 1. Cumulated amount of lidocaine from Example 9 in the in-vitro skin permeation study during 12 hours was 229.94±49.88 (21.7%) μg/cm².

### EXAMPLE 10

An adhesive matrix composed of lidocaine 5.0% (w/w), Lauroglycol 3.0% (w/w), Talc 30.0% (w/w) and Gelva 737 adhesive solution (32.3% polyacrylate) was prepared according to the manufacturing procedures as described in Example 1. Cumulated amount of lidocaine from Example 10 in the in-vitro skin permeation study during 12 hours was 238.76±21.08 (8.8%) μg/cm².

### EXAMPLE 11

An adhesive matrix composed of lidocaine 5.0% (w/w), lauroglycol 4.0% (w/w), Talc 30.0% (w/w) and Gelva 737 adhesive solution (32.3% polyacrylate) was prepared according to the manufacturing procedures as described in Example 1. Cumulated amount of lidocaine from Example 11 in the in-vitro skin permeation study during 12 hours was 197.81±31.64 (16.0%) μg/cm².

### EXAMPLE 12

An adhesive matrix composed of lidocaine 5.0% (w/w), Lauroglycol 2.0% (w/w), silica gel 1.50% (w/w), magnesium stearate 35.0% (w/w) and Gelva 737 adhesive solution (32.3% polyacrylate) was prepared according to the manufacturing procedures as described in Example 1. Cumulated amount of lidocaine from Example 12 in the in-vitro skin permeation study during 12 hours was 207.76±38.37 (18.5%) μg/cm².

### EXAMPLE 13

An adhesive matrix composed of lidocaine 5.0% (w/w), propylene glycol 10.0% (w/w) and Duro-tak 87-2852 adhesive solution (35.0% polyacrylate) was prepared according to the manufacturing procedures as described in Example 1. Cumulated amount of lidocaine from Example 13 in the in-vitro skin permeation study during 12 hours was 79.22±7.76 (9.8%) μg/cm².

### EXAMPLE 14

An adhesive matrix composed of lidocaine 5.0% (w/w), propylene glycol 5.0% (w/w) and Duro-tak 87-2852 adhesive solution (35.0% polyacrylate) was prepared according to the manufacturing procedures as described in Example 1. Cumulated amount of lidocaine from Example 14 in the in-vitro skin permeation study during 12 hours was 53.73±4.59 (8.6%) μg/cm².

### EXAMPLE 15

An adhesive matrix composed of lidocaine 5.0% (w/w), Lauroglycol 10.0% and Duro-tak 87-2852 adhesive solution (35.0% polyacrylate) was prepared according to the manufacturing procedures as described in Example 1. Cumulated amount of lidocaine from Example 15 in the in-vitro skin permeation study during 12 hours was 36.18±2.43 (6.7%) μg/cm².

### EXAMPLE 16

An adhesive matrix composed of lidocaine 5.0% (w/w), propylene glycol 5.0%, lauroglycol 5.0% (w/w) and Duro-tak 87-2852 adhesive solution (35.0% polyacrylate) was prepared according to the manufacturing procedures as described in Example 1. Cumulated amount of lidocaine from Example 16 in the in-vitro skin permeation study during 12 hours was 33.81±0.88 (2.6%) μg/cm².

### EXAMPLE 17

An adhesive matrix composed of lidocaine 5.0% (w/w), propylene glycol 2.0%, Tween-80 3.0% (w/w), lauroglycol 5.0% (w/w) and Duro-tak 87-2852 adhesive solution (35.0% polyacrylate) was prepared according to the manufacturing procedures as described in Example 1. Cumulated amount of lidocaine from Example 17 in the in-vitro skin permeation study during 12 hours was 46.58±15.33 (32.9%) μg/cm².

### EXAMPLE 18

An adhesive matrix composed of lidocaine 5.0% (w/w), TiO2 55.0% (w/w) and Gelva 737 adhesive solution (32.3% polyacrylate) was prepared according to the manufacturing procedures as described in Example 1. Cumulated amount of lidocaine from Example 18 in the in-vitro skin permeation study during 12 hours was 203.45±48.37 (23.8%) μg/cm².

### EXAMPLE 19

An adhesive matrix composed of lidocaine 5.0% (w/w), lauroglycol 2.0%, TiO2 60.0% (w/w) and Gelva 737 adhesive solution (32.3% polyacrylate) was prepared according to the manufacturing procedures as described in Example 1. Cumulated amount of lidocaine from Example 19 in the in-vitro skin permeation study during 12 hours was 215.59±20.31 (9.4%) μg/cm².

### EXAMPLE 20

An adhesive matrix composed of lidocaine 5.0% (w/w), propylene glycol 2.0% (w/w), TiO2 55.0% (w/w) and Gelva 737 adhesive solution (32.3% polyacrylate) was prepared according to the manufacturing procedures as described in Example 1. Cumulated amount of lidocaine from Example 20 in the in-vitro skin permeation study during 12 hours was 250.22±88.12 (35.2%) μg/cm².

### EXAMPLE 21

An adhesive matrix composed of lidocaine 5.0% (w/w), magnesium stearate 55.0% (w/w) and Gelva 737 adhesive solution (32.3% polyacrylate) was prepared according to the manufacturing procedures as described in Example 1.

### EXAMPLE 22

An adhesive matrix composed of lidocaine 5.0% (w/w), magnesium stearate 60.0% (w/w) and Gelva 737 adhesive solution (32.3% polyacrylate) was prepared according to the manufacturing procedures as described in Example 1.

### EXAMPLE 23

An adhesive matrix composed of lidocaine 5.0% (w/w), talc 55.0% (w/w) and Gelva 737 adhesive solution (32.3% polyacrylate) was prepared according to the manufacturing procedures as described in Example 1.

### EXAMPLE 24

An adhesive matrix composed of lidocaine 5.0% (w/w), talc 60.0% (w/w) and Gelva 737 adhesive solution (32.3% polyacrylate) was prepared according to the manufacturing procedures as described in Example 1.

### EXAMPLE 25

An adhesive matrix composed of lidocaine 5.0% (w/w), starch 55.0% (w/w) and Gelva 737 adhesive solution (32.3% polyacrylate) was prepared according to the manufacturing procedures as described in Example 1.

### EXAMPLE 26

An adhesive matrix composed of lidocaine 5.0% (w/w), starch 60.0% (w/w) and Gelva 737 adhesive solution (32.3% polyacrylate) was prepared according to the manufacturing procedures as described in Example 1.

### EXAMPLE 27

An adhesive matrix composed of lidocaine 5.0% (w/w), sorbitol 55.0% (w/w) and Gelva 737 adhesive solution (32.3% polyacrylate) was prepared according to the manufacturing procedures as described in Example 1.

### EXAMPLE 28

Mix Gelva 737 adhesive solution 80.0% (w/w) and Duro-Tak 87-2852 adhesive solution 20.0%. (w/w) together until homogeneous to construct an adhesive solution, Adhesive Solution A.

### EXAMPLE 29

An adhesive matrix composed of lidocaine 5.0% (w/w), talc 20.0% (w/w), starch 40.0% (w/w) and Adhesive Solution A 35.0% was prepared according to the manufacturing procedures as described in Example 1.

### EXAMPLE 30

An adhesive matrix composed of lidocaine 5.0% (w/w), silicon dioxide 1.0% (w/w), talc 20.0% (w/w), starch 39.0% (w/w) and Adhesive Solution A 35.0% was prepared according to the manufacturing procedures as described in Example 1.

### EXAMPLE 31

An adhesive matrix composed of lidocaine 5.0% (w/w), silicon dioxide 2.0% (w/w), talc 20.0% (w/w), starch 38.0% (w/w) and Adhesive Solution A 35.0% was prepared according to the manufacturing procedures as described in Example 1.

### EXAMPLE 32

An adhesive matrix composed of lidocaine 5.0% (w/w), magnesium 20.0% (w/w), starch 40.0% (w/w) and Adhesive Solution A 35.0% was prepared according to the manufacturing procedures as described in Example 1.

### EXAMPLE 33

An adhesive matrix composed of lidocaine 5.0% (w/w), silicon dioxide 1.0% (w/w), magnesium stearate 20.0% (w/w), starch 39.0% (w/w) and Adhesive Solution A 35.0% was prepared according to the manufacturing procedures as described in Example 1.

### EXAMPLE 34

An adhesive matrix composed of lidocaine 5.0% (w/w), silicon dioxide 2.0% (w/w), magnesium stearate 20.0% (w/w), starch 38.0% (w/w) and Adhesive Solution A 35.0% was prepared according to the manufacturing procedures as described in Example 1.

### EXAMPLE 35

Mix Gelva 737 adhesive solution 70.0% (w/w) and Duro-Tal 87-2852 adhesive solution 30.0% (w/w) together until homogeneous to construct an adhesive solution, Adhesive Solution B.

### EXAMPLE 36

An adhesive matrix composed of lidocaine 5.0% (w/w), talc 20.0% (w/w), starch 40.0% (w/w) and Adhesive Solution B 35.0% was prepared according to the manufacturing procedures as described in Example 1.

### EXAMPLE 37

An adhesive matrix composed of lidocaine 5.0% (w/w), silicon dioxide 1.0% (w/w), talc 20.0% (w/w), starch 39.0% (w/w) and Adhesive Solution B 35.0% was prepared according to the manufacturing procedures as described in Example 1.

### EXAMPLE 38

An adhesive matrix composed of lidocaine 5.0% (w/w), silicon dioxide 2.0% (w/w), talc 20.0% (w/w), starch 38.0% (w/w) and Adhesive Solution B 35.0% was prepared according to the manufacturing procedures as described in Example 1.

### EXAMPLE 39

An adhesive matrix composed of lidocaine 5.0% (w/w), magnesium stearate 20.0% (w/w), starch 40.0% (w/w) and Adhesive Solution B 35.0% was prepared according to the manufacturing procedures as described in Example 1.

### EXAMPLE 40

An adhesive matrix composed of lidocaine 5.0% (w/w), silicon dioxide 1.0% (w/w), magnesium stearate 20.0% (w/w), starch 39.0% (w/w) and Adhesive Solution B 35.0% was prepared according to the manufacturing procedures as described in Example 1.

### EXAMPLE 41

An adhesive matrix composed of lidocaine 5.0% (w/w), silicon dioxide 2.0% (w/w), magnesium stearate 20.0% (w/w), starch 38.0% (w/w) and Adhesive Solution B 35.0% was prepared according to the manufacturing procedures as described in Example 1.

### EXAMPLE 42

An adhesive matrix composed of tolterodine 6.0% (w/w) and Gelva 737 adhesive solution (32.3% polyacrylate) was prepared according to the manufacturing procedures as described in Example 1. Cumulated amount of tolterodine from Example 42 in the in-vitro skin permeation study during 24 hours was 176.4±14.1 (8.0%) μg/cm².

### EXAMPLE 43

An adhesive matrix composed of tolterodine 6.0% (w/w), oleyl alcohol 2.0% and Gelva 737 adhesive solution (32.3% polyacrylate) was prepared according to the manufacturing procedures as described in Example 1. Cumulated amount of tolterodine from Example 43 in the in-vitro skin permeation study during 24 hours was 122.2±23.4 (19.1%) μg/cm².

What is claimed is:

1. A transdermal patch comprising:
a backing layer;
an adhesive drug matrix having a pharmaceutically active ingredient and at least one pharmaceutically inactive ingredient; and
a release liner.

2. The patch of claim 1, wherein the adhesive drug matrix further comprises a pressure sensitive adhesive.

3. The patch of claim 2, wherein the pharmaceutically active ingredient is an oil at room temperature.

4. The patch of claim 3, wherein the pharmaceutically active ingredient is selected from the group consisting of lidocaine, oxybutynin, rivastigmine, tolterodine, and combinations thereof.

5. The patch of claim 2, wherein the pharmaceutically active ingredient has a melting point of less than about 80° C.

6. The patch of claim 1, wherein the pharmaceutically active ingredient is a vitamin.

7. The patch of claim 1, wherein the pharmaceutically inactive ingredient is selected from the group consisting of talc, titanium dioxide, silica gel, magnesium stearate, starch, dextrose and sorbitol.

8. The patch of claim 2, wherein the adhesive is a polyacrylate polymer.

9. The patch of claim 4, wherein the rivastigmine has about 1% to about 10% by weight of the adhesive drug matrix.

10. The patch of claim 4, wherein the rivastigmine has about 1% to about 10% by weight of the adhesive drug matrix.

11. The patch of claim 4, wherein the rivastigmine has about 1% to about 10% by weight of the adhesive drug matrix.

12. The patch of claim 4, wherein the rivastigmine has about 1% to about 10% by weight of the adhesive drug matrix.

13. The patch of claim 7, wherein the pharmaceutically inactive ingredient has about 20% to about 80% by weight of the adhesive drug matrix.

14. The patch of claim 1, wherein the adhesive drug matrix comprises about 0.1 to about 10% of an active ingredient (w/w) and at least one pharmaceutically inactive ingredient in addition to the adhesive.

15. The patch of claim 1, wherein the adhesive drug matrix has adhesive and pharmaceutically active ingredient in an amount of about 0.5% to about 15%.

16. A method for preparation of patch comprising:
mixing a predetermined amount of least one pharmaceutically active ingredient with predetermined amount at least one pharmaceutically inactive ingredient to form a mixture;
adding the mixture into a solution with an adhesive;
mixing the solution until it becomes homogenous;
film coating a release liner with the homogeneous solution; and
laminating the film coated release liner to produce a transdermal patch.

17. The method of claim 16, wherein the transdermal patch is cut into desired sizes.

18. A method of treating a mammal in need of treatment, the method comprising applying the transdermal patch of claim 1 to the skin of the mammal.
